(19) United States
(12) Patent Application Publication
(10) Pub. No.: US 2005/0136507 A1
(43) Pub. Date: Jun. 23, 2005

(54) Portable disposable airborne pathogen collection device and system

(76) Inventors: George D. Sullivan, Evanston, IL (US); James F. Sullivan, Park Ridge, IL (US); Daniel J. Sullivan, Evanston, IL (US)

Correspondence Address: VEDDER PRICE KAUFMAN & KAMMHOLZ, 222 N. LASALLE STREET, CHICAGO, IL, 60601, US

(21) Appl. No.: 10/742,293

(22) Filed: Dec. 19, 2003

(51) Int. Cl. C12Q 1/24

(52) U.S. Cl. 435/30; 435/309.1

(57) ABSTRACT

An apparatus for the collection of airborne microorganisms and gaseous substances includes an air intake mechanism capable of drawing in an air sample. The air sample is thereupon percolated through a reservoir containing a liquid means as the air is passed through an air intake chamber and through the reservoir. As the air sample is passed therethrough, airborne pathogens including microorganisms or gaseous substances disposed within the air sample will become suspended within the liquid means. The air sample is thereupon exhausted through the apparatus through an exhaust chamber. A sampling port adjacent to the reservoir allows for the extraction of a sample of liquid means so the sample may be tested to determine the presence of any airborne pathogens including microorganisms or gaseous substances by means of its DNA or genetic fingerprint or by gas mass spectrometry.

### FIELD OF THE INVENTION

The present invention relates generally to the capture and detection of airborne, infectious microorganisms and poisonous effluvia in indoor air and more specifically to a portable disposable airborne microorganism collection device and system constructed so that “DNA” or other genetic identification can be made specific to allow the proper prescription of medicines for victims, and notification to public.

### BACKGROUND OF THE INVENTION

Due to recent warfare and to the outbreak of highly infectious diseases attributable to airborne microorganisms and toxic gases in indoor air, an urgent need has arisen for the rapid collection and detection of these infectious microorganisms, and gases such as viruses, bacteria, mold, yeast and spores, and gaseous substances. Typically, larger particles fall to the floor or other surfaces and these become dust-associated mycobacterial particles and are not believed to pose a serious health risk due to their large size. The smaller mycobacterial-containing particles, and gaseous substances however, undergo rapid evaporation and may remain airborne indefinitely. These gases and sub-micron size particles pose a serious risk of absorption and respiratory track infection and disease.

There is now a special war-time footing need for detecting and collecting weapons of mass destruction (WMD) and infectious gases and airborne microorganisms for rapid identification. Diseases caused by the airborne microorganisms and gases may be readily spread over entire cities and between persons located in indoor proximity, such as barracks, prisons, hospitals, shelters, classrooms, subways, trains, aircraft and the workplace or any other indoor location.

Currently, there exists methods for detecting and collecting infectious airborne microorganisms and gases for rapid identification (but without immediate verification ability) involving large detection devices having significant size and limited portability. For example, the airborne microorganisms collection apparatus disclosed by U.S. Pat. No. 5,766,958 discloses an airborne microorganism collection device with a large intake device for percolating an air sample through a liquid. Further, the device causes the collection of mist from the percolated liquid and a sample is drawn from the collected condensation from the mist. The system described in U.S. Pat. No. 5,766,958 also provides for allowing the recycling of the liquid because standard laboratory procedure requires any detection of airborne pathogens should be retested to confirm the results and especially so with WMD. Therefore, the apparatus cannot be fully cleaned and the liquid recycled such that another laboratory required conformation test may be performed immediately without concern of contamination from previous tests. The United States Code of Federal Regulations 32 CFR § 627 et seq. sets forth stringent rules for decontamination where sterility is required.

Another currently available air sampling device is an air collection device manufactured by Septor Industries, Inc. of 4950 Cherry, Kansas City, Mo. 64110. One current sampler manufactured by Septor Industries, Inc. is entitled the SpinCon® which has an approximate weight of 46 pounds (without attachments) and a dimension of 18 inches by 15 inches by 8 inches. The collection device is housed within a portable carrying case and allows for a one-time detection usage. Spincon's own operating website rules require disposal of its unit if contaminated by a “hit” in any WMD ASSAT capture. Therefore, if an initial positive identification is found, a second detection device must be implemented for the immediate confirmation of the original test.

Another commonly used protective device is the so-called “high efficiency” particulate air (HEPA) filter. This device may be unsatisfactory because it may become clogged with living breeding microorganisms and offers little or no protection from microorganisms which are smaller than its smallest orifice. These pathogenic microorganisms may bypass the HEPA filter as well as nasal and throat defenses and reach the lungs of an individual. Also, the HEPA filter is useful if the microorganisms are to be cultured, but many airborne pathogens are not; and this procedure is time-consuming and an outdated identification procedure.

An alternative to the HEPA filter is a system which kills, but does not collect pathogenic microorganisms by subjecting them to ultraviolet radiation. This system is not widely used, however, because it exposes workers and others to potentially harmful radiation. A room RIR sterilizer, such as disclosed in U.S. Pat. No. 5,225,167, combines the HEPA filter with a germicidal ultraviolet lamp. The lamp is positioned to as to kill the microorganisms trapped in the HEPA filter without exposing workers and others to radiation. This system does not collect microorganisms for subsequent identification and offers no protection from sub-micron sized microorganisms which are small enough to pass through the smallest orifices of the HEPA filter. For example, many viruses are only 300 to 400 angstroms in size, much smaller than the current orifices of many HEPA filters. Other alternatives are the impacters and impingers. An impacter drives diseased air against a series of sampling plates having successively smaller holes. The largest microorganisms are collected at the first stage and the smaller microorganisms are collected at later stages. While impacters collect microorganisms for identification, they like the HEPA filter, suffer from drawbacks of allowing sub-micron size organisms to escape.

The liquid impinger employs a compressor to draw diseased air at nearly sonic velocity into a liquid medium. This methodology, however, disintegrates many cells, which distorts and impedes the detection and collection of the pathogens, particularly those of less than one micron in size. Moreover, the necessity of a compressor renders a liquid impinger impractical for hospital use.

As such, there remains an urgent wartime need for detecting the presence of airborne infectious microorganisms and gases in indoor air and collecting those microorganisms and effluvium simultaneously for rapid identification using a devices that can be easily positioned for airborne pathogen and gaseous substance detection and allows for quick efficient secondary recapture for retesting to verify previous results.

### SUMMARY OF THE INVENTION

The present invention is a multiple portable disposable airborne pathogen and gaseous collection device and system including an air intake mechanism capable of drawing an air sample. The air intake mechanism may be a fan capable of drawing the air sample from an indoor area into the collection apparatus. The collection device further includes an air intake chamber which is coupled to the air intake mechanism such that the air sample is passed through the air intake chamber to a reservoir containing a liquid means.

The air sample is then percolated through the liquid means such that any airborne pathogens and gaseous substances within the air sample becomes suspended within the liquid means. The portable disposable airborne collection device further includes an exhaust chamber coupled to the reservoir so that the air sample, upon passing through the liquid means passes out through the exhaust chamber. The air sample passes out of the exhaust chamber without a sizeable portion of the previously airborne pathogens and gaseous substances, which are suspended within the liquid means. Further included is a sampling port which is adjacent to the reservoir so that a sample of the liquid means may be extracted.

The extracted sample may then be tested by DNA, genetic testing, or a gas chromatograph to determine the precise airborne pathogens or gases which are suspended within the liquid means. Once an airborne pathogen or gas is found within the liquid means, or after a predetermined period of time in which the liquid means has had air samples percolated therethrough, this small economic airborne pathogen and gas collection device may be discarded as required by Federal Rules (32 CFR § 627 et seq.). Another duplicate portable disposable airborne collection device immediately at hand in the same transporting “pack” may be utilized to retest another air sample for the purpose of verifying the presence of bio-chem WMD.

A unique solution to the crucial necessity of immediate verification of the precise gaseous or DNA so-called genetic “fingerprint”, almost immediately following the first positive particulate or gaseous assay is provided by the multiplicity of lightweight devices, delivered to a test site in multiple numbers in the same transportation “pack” or carrying case. The momentary change in the number and morphology of a pathogen or the dissipation of a gas within moments of a first sample can be essential to the determination and defeat (through the proper defense or medical prescription) of the threat to life posed in a given terrorist attack. The infinitesimally small concentration in a sub-micro size of a given pathogen and the rapid dissipation of the strength of a gaseous substance demand immediate confirmation retesting. The means must be immediately at hand together with exact duplicate liquids in place and with battery or back-up power supply to conduct such testing. Indeed, if the period of gestation of a known or unknown microorganism pathogen upon the human or animal species is very brief, the immediacy of verification and diagnosis may spell life or death to the victim of inhalation or skin absorption. The reproduction, therefore, of the immediate and exact measurement of the WMD agent presents an unique necessity for the multiple pack assembly of diagnostic equipment described herein, which is designed to repeat and quickly confirm or deny the threat to life.

### BRIEF DESCRIPTION OF THE DRAWINGS

The invention will be better understood with reference to the following drawings wherein:

FIG. 1 illustrates a side view of a portable disposable airborne collection device, in accordance with one embodiment of the present invention;

FIG. 2 illustrates a prospective view of the airborne collection device;

FIG. 3 illustrates a front view of the airborne collection device, in accordance with one embodiment of the present invention;

FIG. 4 illustrates an alternative embodiment of a portable and disposable airborne collection device; and

FIG. 5 illustrates a cross section of the portable disposable airborne pathogen collection device of FIG. 4 cross section V-V.

### DETAILED DESCRIPTION OF PREFERRED EMBODIMENTS

An apparatus for the simultaneous collection of airborne microorganisms and gaseous substances 100 includes an air intake mechanism 102, which is capable of drawing in an air sample. In one embodiment, the air intake mechanism is a rotatable fan, which may be battery operated or powered by any other means such that the fan 102 rotates and draws the air sample therein. The apparatus 100 further includes an air intake chamber 104 disposed between the air intake mechanism 102 and a reservoir 106 that contains a liquid means 108.

In one embodiment, the apparatus 100 is made of a composite formed hard plastic material forming the cavities that define the air intake chamber 104 and the reservoir 106 containing the liquid means 108. Moreover, the liquid means 108 may be any available liquid capable of having an air sample percolated therethrough and supporting the suspension of one or more previously airborne pathogens, such as distilled water, a liquid disinfectant, or any other suitable liquid capable of such suspension of gaseous substances suspending, as recognized by one having ordinary skill in the art.

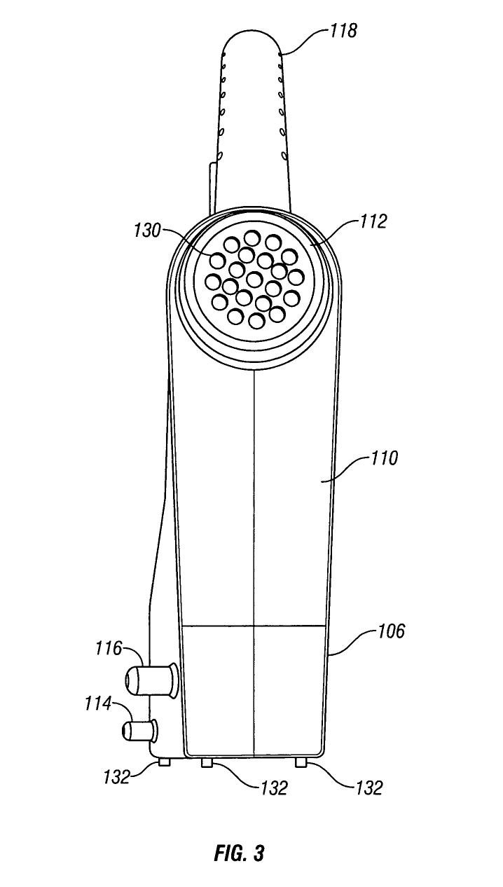

The apparatus 100 further includes an exhaust chamber 110 disposed above the reservoir 106 accessible through the liquid means 108 within the reservoir 106. Therefore, an air sample drawn in through the air intake 102, down through the air intake chamber 104, and percolated through the liquid means 108 may be exhausted through an exhaust 112 via the exhaust chamber 110. In one embodiment, the air is drawn at a rate of 15 to 25 cubic feet per minute because any drawing rate greater than 25 cubic feet per minute may disintegrate or otherwise damage any pathogens, thereby impeding and distorting the collection and analysis. As recognized by one having ordinary skill in the art, any suitable air intake mechanism 102 may be utilized to provide for the percolation of the air sample through the liquid means. The draw rate of 15 to 25 cubic feet per minute is based on the liquid means 108 having a specific density of approximately 1.0. As recognized by one having ordinary skill in the art, if the viscosity of the liquid means 108 is adjusted, the rotational speed of the fan may be adequately adjusted to provide the correspondingly appropriate draw rate to preserve the pathogens in the liquid means 108. Furthermore, the exhaust chamber 110 and the exhaust 112 may be defined chambers based on the molded casing of the apparatus 100. Furthermore, the exhaust 112, may contain a perforated cover as illustrated and discussed further below with respect to FIG. 3.

In one embodiment, the apparatus 100 further includes a sampling port 114 adjacent to the reservoir 106 such that a sample of liquid means 108 may be extracted therethrough using extraction means such as a syringe. In one embodiment, the sampling port 114 is a hard plastic extension having a water-tight seal with an opening for inserting a sample extraction device, such as a syringe, therethrough for the extraction of the liquid means 108. In one embodiment, the sampling port 114 is a resealable nipple allowing for the removal of the liquid extraction device and the resealing of the liquid-tightness of the reservoir.

The apparatus 100 further includes liquid fill port 116 and a handle 118. The handle 118 is disposed, in one embodiment, on a posterior position for the ease of portability of the device and the liquid fill port 116 allows for the insertion of the liquid means 108 into the reservoir 106.

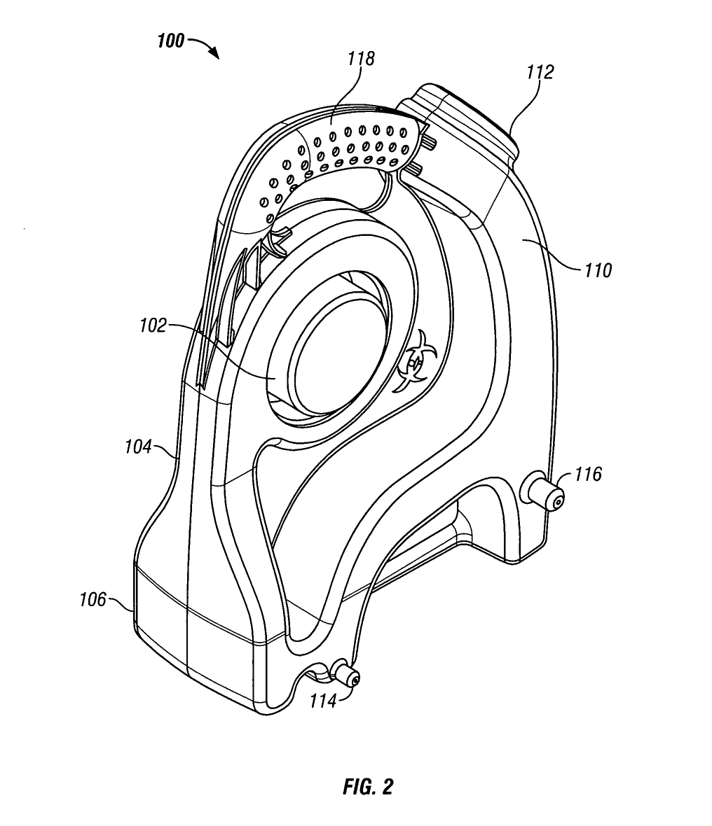

FIG. 2 illustrates a perspective view of the airborne microorganismcollection device 100 better illustrating the perspective alignment of the various elements, including the reservoir 106, the liquid fill port 1 16, the sampling port 114, the air intake chamber 104 and the exhaust chamber 110. Further included in the apparatus 100, not visible in FIG. 2, are a plurality of base footing members disposed on the underside of the apparatus 100 for stabilizing the collection device 100 in an upright position. As better illustrated in FIG. 2, the sampling port 114 and the fill port 116 outwardly extend from the apparatus 100, more specifically extending outward from the reservoir 106, wherein the reservoir 106 extends the full length of the apparatus 100 divided by a portion of the exhaust chamber 110. FIG. 2 also illustrates, in one embodiment, the orientation of the handle 118 relative to the intake mechanism 102 and the exhaust 112.

FIG. 3 illustrates a front view of the apparatus 100 with the exhaust 112 at the exterior of the exhaust chamber 110. In one embodiment, the exhaust 112 includes a perforated cover 130 for allowing the air sample to pass out of the apparatus 100. Further illustrated is the orientation of the handle 118, the outward extensions of the sample port 114 and the filling element 116. Furthermore, the base footing members 132 provide for lateral stability when the device 100 is rested on a flat surface.

As discussed above, in one embodiment, the apparatus 100 may be composed of a hard plastic material, therefore being extremely lightweight and having a great deal of portability. Furthermore, the collection device 100 does not contain a large amount of moving parts and easily disposable after a single use.

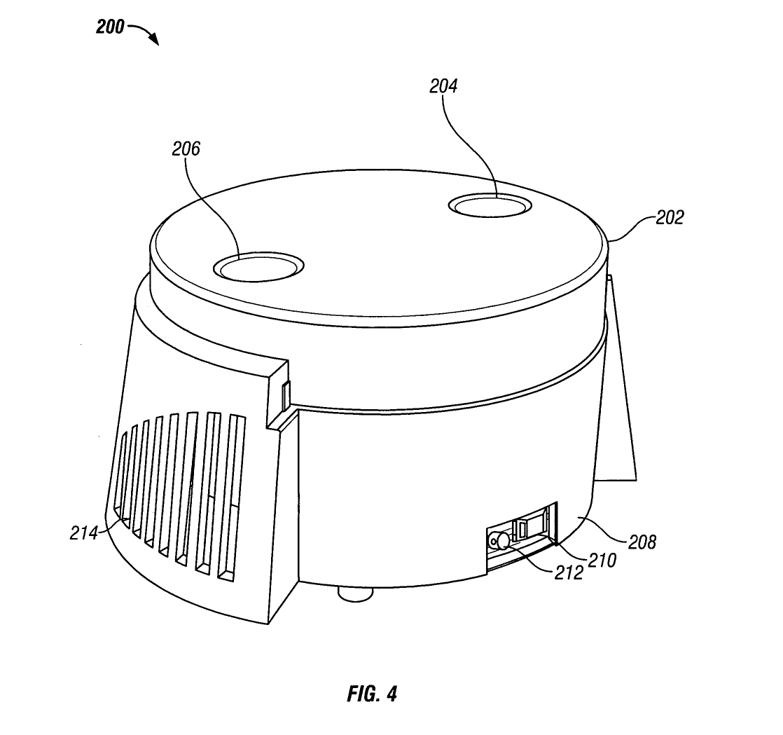

FIG. 4 illustrates another example of an airborne pathogen detection device 200 having a disposable collector reservoir 202 with an air intake port 204 and a sampling port 206. The detection device 200 further includes a base 208 and airflow control means 210. Moreover, in one embodiment, a light indicator 212 is disposed on the base 206 for indicating operation. The airflow control means 210 may be a toggle switch electrically connected to an air intake means, such as a rotary fan (not visible) within the base 208. Furthermore, the base 208 includes a plurality of vertical slots 214, which allow for an intake air sample to be exhausted from the device 200.

FIG. 4 illustrates the exterior portion of the microdetection device 200 portable and disposable. The apparatus 200 allows for the removal of the disposable collector reservoir 202 and the insertion of a new disposable collector reservoir 202 on the base 208 for reuse. As discussed above, the risk for error after first use or reuse resulting in contaminated assays with false negative or positive results is too great to impose upon the public or a first responder. Therefore, a disposable and replaceable collection detection system, such as the system 200, allows for multiple testing using multiple reservoirs without sacrificing critical time lapse, integrity or increasing cost significantly.

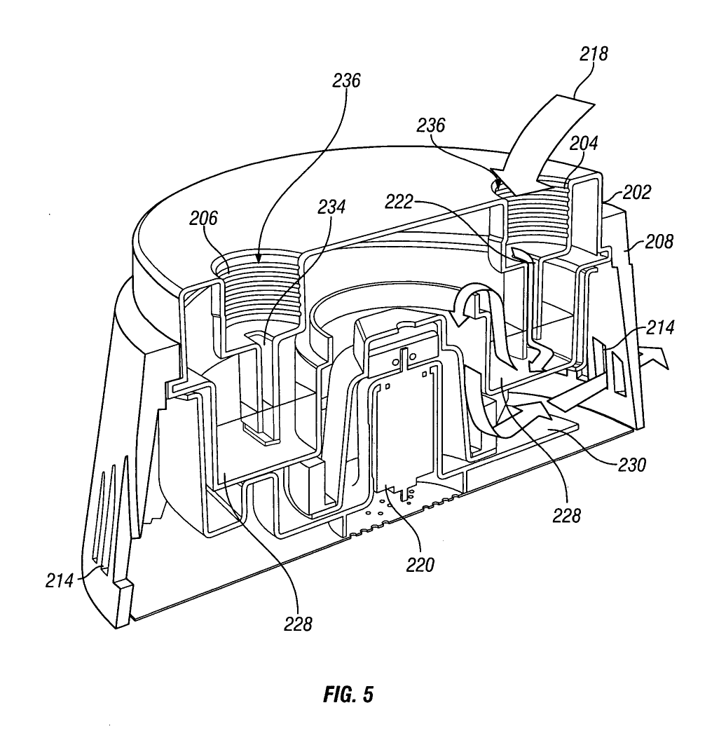

FIG. 5 illustrates a cross-sectional view of the apparatus 200 visible via the cross section V-V of FIG. 4. In the interior of the base 208 is a motor 220, which in one embodiment is sealed off from any airflow 218 and also sealed off from any direct contact with the disposable collector reservoir 202. The motor may be battery operated and by distant electrical command or may be provided with means for rotating an air intake mechanism using any other suitable means, as recognized by one having ordinary skill in the art. The disposable collector reservoir 202 includes an air intake chamber 222 disposed relative to the air intake port 204 such that the motor 220, otherwise referred to as the air intake mechanism, draws an air sample into the air intake chamber 222 through the port 204.

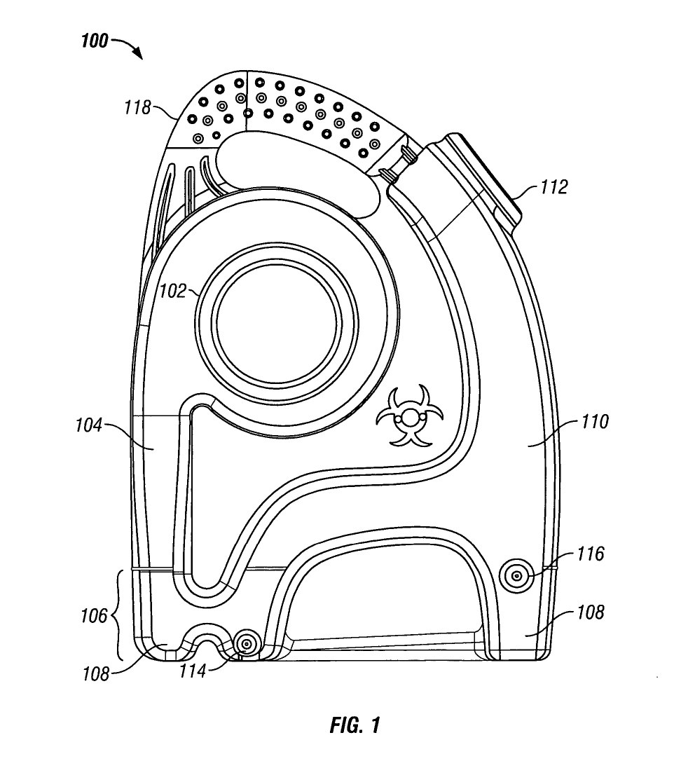

Within the disposable collector reservoir 200 is a reservoir 226 which contains a liquid means 228, similar to the liquid means 106 with respect to FIGS. 1-3. The reservoir 226 containing the liquid means 228 is disposed relative to the air intake mechanism 220 such that the air sample 218 is drawn through the air intake chamber 222 and percolated through the liquid means 228. Thereupon, airborne pathogens and/or gaseous substances within the air sample 224 become suspended in the liquid means 228. Similar to the embodiment of FIGS. 1-3, the air intake mechanism 220 operates to draw the air through the intake port 222 at a predefined air flow rate. Q: What is air flow rate range? 15-25 CFM

The apparatus 200 further includes an exhaust chamber 230 which extends circumferentially around the base 208 such that the air sample 210 drawn through the liquid means 228, upon percolation, enters into the exhaust chamber 230 and passes through the exhaust slots 214. The exhaust chamber 230 circumferentially extends around the motor 220, as does the reservoir 226 and the liquid means 228 contain therein. As an air sample 218 is percolated through one side of the reservoir 226, in one embodiment on the opposing side, the sampling port 206 allows for the extraction of a sample of the liquid means 228. More specifically, the sampling port 206 includes a sample chamber 234 such that a sampling device, such as a syringe, may be projected down into the channel 234 through the sampling port 206 for liquid extraction.

In one embodiment, the air intake port 204 and the sampling port 206 may further include a threading 236 which allows for a plug (not shown) to be inserted therein. Therefore, the collector reservoir 202 may be sealed using the plugs insuring the integrity of the interior of the collector reservoir 204, including the liquid means 208 during shipment.

In this embodiment, upon using the disposable collector reservoir 202 for obtaining a liquid sample, the disposable collector reservoir 202 may be readily withdrawn from the base 206 and a new disposable collector reservoir 202 disposed thereon. Therefore, through the easy replacement of different disposable collector reservoirs 202 relative to a single base 206, multiple testings can be performed with the disposable collector reservoir 202 being readily disposable without added cost due to reusing the base 206. Furthermore, upon sampling the ports 204 and 206 may be sealed such that any potentially contaminated liquid means may be properly disposed of.

The portable disposable airborne collection device may further be incorporated into a system for airborne detection. As discussed above, in order to verify and maintain a preferred accuracy level of airborne detection, a test must be repeated more than one time. Therefore, in the event a first positive test result confirms the presence of airborne pathogens, or a noxious gaseous substance, another test must be performed. Thus, a system for airborne pathogen collection includes, in one embodiment, a plurality of disposable airborne pathogen detection devices, such as 100 of FIG. 1 or 202 of FIG. 4. When the system includes multiple devices 100 or 202, a first device may be operated in accordance with the devices as described above. Upon the extraction of a sample and the testing of the sample, a second device may be implemented to perform a second test.

Therefore, in one embodiment a first apparatus 100 of FIG. 1 may be used to test a first air sample pulled in through the air intake means 102. Upon being percolated through the liquid means 108, the air sample may be exhausted through the exhaust chamber 110. Therefore, a first sample may be extracted using a first sampling port 114.

In order to assure accuracy, a duplicate second device to the device 100 of FIG. 1, may be utilized having a second air intake mechanism, a duplicate of the air intake mechanism 102 of FIG. 1. A second air sample is pulled in through the second air intake chamber of the second collection device and percolated through a second liquid means disposed within a second reservoir. The second air sample may then be exhausted through a second exhaust chamber. Moreover, the second collection device may further include a second sampling port, similar to port 114 of FIG. 1, so that a second sample of the liquid means may be extracted and tested. The test results of the first sample of liquid means may then be compared with the second sample of liquid means to verify the accuracy of the test results.

In the above embodiment with the device 100, a plurality of the devices 100 may be utilized wherein the devices are extremely portable and may be readily disposed of upon usage. Although, in the second embodiment of the present invention, such as the apparatus 200 illustrated in FIG. 4, a single base unit 208 may be provided having a plurality of the disposable collection reservoir 202. Upon the testing of the first air sample 218 and the extraction through the sampling port 234, a first disposable collection reservoir 202 may be removed from the base 208 and a second disposable collection reservoir, a duplicate of the disposable collection reservoir 202 of FIG. 4, may be disposed on the base 208. Therefore, the toggle switch 210 may be reactivated, thereby reactivating the air intake mechanism 220 to percolate another air sample through liquid means disposed within the second reservoir. In this embodiment, the first air intake mechanism is the same as the second air intake mechanism 220 disposed within the base 208 and is reusable for each sample because the air intake mechanism 220 is sealed off from the liquid means and therefore is not subject to contamination.

In furtherance with the collection of a sample of the liquid means, the sample may be tested using a laboratory. In one embodiment, based on the mobility of the collection device 100 or 200, a mobile laboratory may be utilized for immediate testing at an on-site location. For example, an Agilent Mobile Laboratory may be utilized to perform analytical measurement system within a mobile laboratory to detect and confirm the presence of chemical and biological agents. The mobile laboratory is available for purchase from Government Scientific Source (GSS), an Agilent Channel Partner, on GSA contract number GS-24F-1181B, Part No. MLP28TK.

Whether the testing is performed in a mobile laboratory or other testing device, using any available testing techniques, the liquid means sample is tested for the presence of airborne pathogens including microorganisms. For example, mass spectrometry may be utilized to perform spectromatic testing. In another embodiment, chromatography may be utilized to test the liquid sample. Regardless thereof, on the extraction of a liquid means sample, a liquid means sample may be tested using any commonly available or known testing system such as a “PCR” genetic detector, which allows for the verification or authentication of airborne pathogens or microorganism suspended within the liquid means.

It should be understood that there exists implementations of other variations and modifications of the invention and its various aspects, as may be readily apparent to those of ordinary skill in the art, and that the invention is not limited by the specific embodiments described herein. For example, the apparatus 100 or device 202 may be composed of any readily available material allowing for the formation of the defined air passage chambers and liquid means holding reservoir, wherein the material allows for easy portability and disposability. It is therefore contemplated and covered by the present invention, any and all modifications, variations, or equivalents that fall within the scope of the basic underlying principles disclosed and claimed herein.

1. An apparatus for the collection of at least one of an airborne microorganisms and gaseous effluvia, the apparatus comprising:
an air intake mechanism capable of drawing an air sample;
an air intake chamber operatively coupled to the air intake mechanism such that the air sample is passed therethrough;
a reservoir containing a liquid means, the reservoir disposed relative to the air intake chamber such that the air sample passes through the reservoir and is percolated through the liquid means such that airborne microorganisms or gaseous effluvia within the air sample becomes suspended in the liquid means;
an exhaust chamber operatively coupled to the reservoir such that the air sample, upon passing through the liquid means, passes through the exhaust chamber; and
a sampling port adjacent to the reservoir such that a sample of the liquid means may be extracted therethrough.

2. The apparatus of claim 1 wherein the air intake mechanism is a motor-powered fan.

3. The apparatus of claim 1 further comprising:
a carrying handle such that the apparatus is mobile.

4. The apparatus of claim 1 further comprising:
a liquid fill port adjacent to the reservoir such that the reservoir may be provided with the microorganism or gaseous substance suspension liquid through the liquid fill port.

5. The apparatus of claim 1 further comprising:
a plurality of stabilizing feet disposed on a bottom side of the apparatus.

6. The apparatus of claim 1 wherein the air intake chamber, the reservoir and the exhaust chamber are defined by a plastic casing, wherein the plastic casing is disposable.

7. The apparatus of claim 1 wherein the liquid means is at least one of the following: distilled water and liquid disinfectant:
wherein the plastic casing is positioned together in a carrying case with at least two duplicate tubes therewith.

8. A method for the collection of at least one of airborne microorganisms and gaseous substances, the method comprising:
drawing an air sample into an air intake chamber of a portable airborne pathogen and gaseous substances collection device using an air intake mechanism;
providing the air sample to a collection reservoir;
percolating the air sample through a liquid means disposed within the collection reservoir;
exhausting the air sample out through an exhaust chamber, wherein airborne microorganisms and gaseous substances are extracted simultaneously from the air sample and suspended in the liquid means during percolation; and
providing a portion of the liquid means directly from the collection reservoir to a sampling port coupled to the reservoir for the extraction of a sample of the liquid means;
allowing for the extraction of a liquid means sample such that the liquid means sample may be tested; and
discarding the portable airborne pathogen detection device.

9. The method of claim 8 wherein a motor-powered fan draws the air sample into the air intake chamber.

10. The method of claim 8 further comprising:
filling the reservoir with the liquid means via a liquid fill port coupled to the reservoir.

11. The method of claim 8 wherein the air intake chamber, the reservoir and the exhaust chamber of the detection device are defined by a plastic casing such that the plastic casing is disposable.

12. The method of claim 8 wherein the liquid means is at least one of the following: distilled water and liquid disinfectant or gaseous and particulate agglomerate.

13. A method for collecting a liquid sample in a portable airborne microorganism and gaseous substance collection device, the method comprising:
providing the portable airborne microorganism and gaseous substance collection device including:
an air intake mechanism capable of drawing an air sample;
an air intake chamber operatively coupled to the air intake mechanism such that the air sample is passed therethrough;
a reservoir containing a liquid means, the reservoir disposed relative to the air intake chamber such that the air sample passes through the reservoir and is percolated through the liquid means such that an airborne pathogen and gaseous substance within the air sample becomes suspended in the liquid means;
an exhaust chamber operatively coupled to the reservoir such that the air sample, upon passing through the liquid means, passes through the exhaust chamber; and
a sampling port adjacent to the reservoir such that a sample of the liquid means may be extracted therethrough;
extracting a sample from the sample port; and
discarding the portable airborne microorganism and gaseous substance collection device.

14. The method of claim 13 wherein the portable airborne microorganism and gaseous substance collection device further includes:
a carrying handle such that the apparatus is mobile.

15. The method of claim 13 wherein the portable airborne microorganism detection device further includes:
a liquid fill port adjacent to the reservoir such that the reservoir may be provided with the pathogen suspension liquid through the liquid fill port.

16. The method of claim 13 wherein the portable airborne collection device further includes:
a plurality of stabilizing feet disposed on a bottom side of the apparatus.

17. The method of claim 13 wherein the air intake chamber, the reservoir and the exhaust chamber are defined by a disposable plastic casing.

18. The method of claim 13 wherein the liquid means is at least one of the following: distilled water and liquid disinfectant and gaseous or particulate agglomerate.

19. A portable disposable apparatus for the collection of at least one of airbornemicroorganisms and gaseous substances, the apparatus comprising:
a motor powered fan capable of drawing an air sample;
an air intake chamber operatively coupled to the air intake mechanism such that the air sample is passed therethrough;
a reservoir containing a liquid means, the reservoir disposed relative to the air intake chamber such that the air sample passes through the reservoir and is percolated through the liquid means such that an airborne pathogen or gaseous substance within the air sample becomes suspended in the liquid means;
an exhaust chamber operatively coupled to the reservoir such that the air sample, upon passing through the liquid means, passes through the exhaust chamber;
a sampling port adjacent to the reservoir such that a sample of the liquid means may be extracted therethrough;
the air intake chamber, the reservoir, the exhaust chamber and sampling port are defined by a disposable plastic casing and
a readily accessible carrying handle allowing for the ease of portability.

20. The apparatus of claim 19 further comprising:
a liquid fill port adjacent to the reservoir such that the reservoir may be provided with the pathogen suspension liquid through the liquid fill port.

21. The apparatus of claim 1 wherein the liquid means is at least one of the following: distilled water and liquid disinfectant.

22. A system for the collection of at least one of airborne microorganisms and gaseous substances, the system comprising:
a first airborne collection device including:
a first air intake mechanism capable of drawing a first air sample;
a first air intake chamber operatively coupled to the first air intake mechanism such that the first air sample is passed therethrough;
a first reservoir containing a first liquid means, the first reservoir disposed relative to the first air intake chamber such that the first air sample passes through the first reservoir and is percolated through the first liquid means such that an airborne microorganism or gaseous substance within the first air sample becomes suspended in the first liquid means;
a first exhaust chamber operatively coupled to the first reservoir such that the first air sample, upon passing through the first liquid means, passes through the first exhaust chamber; and
a first sampling port adjacent to the first reservoir such that a first sample of the first liquid means may be extracted therethrough; and
a second airborne collection device including:
a second air intake mechanism capable of drawing a second air sample;
a second air intake chamber operatively coupled to the second air intake mechanism such that the second air sample is passed therethrough;
a second reservoir containing a second liquid means, the second reservoir disposed relative to the second air intake chamber such that the second air sample passes through the second reservoir and is percolated through the second liquid means such that an airborne microorganism or gaseous substance within the second air sample becomes suspended in the second liquid means;
a second exhaust chamber operatively coupled to the second reservoir such that the second air sample, upon passing through the second liquid means, passes through the second exhaust chamber; and
a second sampling port adjacent to the reservoir such that a second sample of the liquid means may be extracted therethrough and compared with the first sample.

23. The system of claim 22 wherein, the first air intake mechanism is the same as the second air intake mechanism.

24. The apparatus of claim 1 further comprising a multi-unit carrying case containing a plurality of said apparati.

25. The apparatus of claim 24 further comprising a filler attachment, electrical power or battery inlet, for each.

26. A system for collection of airborne pathogen and gaseous effluvia, said system comprising a plurality of collection device contained in a multipack carrying case.

27. An apparatus for multiple repetitive tests of airborne microorganisms and gaseous substances comprising:
a plurality of air intake chambers individually operatively coupled to a plurality of air intake mechanisms such that the air samples are passed therethrough;
a plurality of reservoirs each containing a liquid means, each of the reservoirs disposed relative to an individual air intake chamber such that the air sample passes through a reservoir and is percolated through the liquid means such that an airborne pathogen or gaseous substance within the air sample becomes suspended in the liquid means;
a plurality of exhaust chambers individually operatively coupled to each of the reservoirs such that the air sample, upon passing through the liquid means, passes through the exhaust chambers;
a plurality of sampling ports, one port adjacent to each of the reservoirs such that a sample of the liquid means may be extracted therethrough; and
the air intake chambers, the reservoirs, the exhaust chambers and sampling ports being defined by one or more disposable plastic casings.

28. The apparatus of claim 27 and further comprising:
a readily accessible carrying handle allowing for the ease of portability of the apparatus.
